(19) United States
(12) Patent Application Publication
(10) Pub. No.: US 2026/0315662 A1
(43) Pub. Date: Oct. 15, 2026

(54) COMPOSITIONS OF MATTER, THAT HAVE ANTIMICROBIAL PROPERTIES, MIXTURES, AND METHODS TO MAKE AND USE THEM

(71) Applicant: Wynnvision, LLC, Midlothian, VA (US)

(72) Inventors: Kenneth WYNNE, Midlothian, VA (US); Olga ZOLOTARSKAYA, Richmond, VA (US); Rebecca JARRELL, Williamsburg, VA (US); Kennard BRUNSON, Henrico, VA (US); Chenyu WANG, Glen Allen, VA (US); Youssef AMIN, Richmond, VA (US)

(21) Appl. No.: 18/881,558

(22) PCT Filed: Dec. 28, 2024
(86) PCT No.: PCT/IB2024/063246

§ 371 Date: Jun. 25, 2026

Related U.S. Application Data
(60) Provisional application No. 63/618,260, filed on Jan. 5, 2024.
(60) Provisional application No. 63/738,804, filed on Dec. 25, 2024.

(51) Int. Cl. A61L 29/04; A01N 33/04; A61L 29/16

(52) U.S. Cl. CPC A61L 29/04; A01N 33/04; A61L 29/16; A61L 2300/204; A61L 2300/404

(57) ABSTRACT

Disclosed are compositions of matter comprising a polymer and an amine that have antimicrobial properties, and mixtures and methods to make and use them.

### CROSS-REFERENCE TO RELATED APPLICATIONS

This application claims priority and benefit from U.S. provisional application 63/618,260, filed Jan. 5, 2024, titled “Antimicrobial Silicones,” and to U.S. provisional application 63/738,804, filed Dec. 25, 2024, and the contents of both of these applications are hereby incorporated herein by reference.

### STATEMENT OF GOVERNMENT INTEREST AND, FEDERAL FUNDING

One or more inventions were made with government support. The contracts or grants that provided funding are NIH 5R44DK103398 and NIH 5R44HL142391. The federal agency that awarded the contracts or grants was the National Institutes of Health. The government has an interest in the invention.

### BACKGROUND OF THE INVENTION

U.S. Pat. No. 11,666,686 issued Jun. 6, 2023, to WynnVision, LLC, the contents of which are hereby incorporated by reference, discloses a composition, comprising: a certain amine and physiologically-acceptable polydimethylsiloxane.

Wynne et al, “Facile Modification of Medical-Grade Silicone for Antimicrobial Effectiveness and Biocompatibility: A Potential Therapeutic Strategy

against Bacterial Biofilms,” ACS Appl. Mater. Interfaces 2023, 15, 46626-46638, the contents of which are hereby incorporated by reference, discloses a one-step modification of biomedical silicone tubing, with N,N-dimethyltetradecylamine.

Herein after “C14” and “C14 amine” mean N,N-dimethyltetradecylamine, CAS Number 112-75-4.

Herein, expressions including “.sub.” or “.sup.” followed by a numeral indicate that the numeral defines subscript or superscript respectively.

### OBJECTS OF THE INVENTION

It is an object to provide novel compositions of matter, manufactures, and machines, that have antimicrobial properties, and methods of making and using them. It is also an object to provide the foregoing that are also human-biocompatible. It is also an object to provide novel antimicrobial products, including implantable medical devices, and methods of their use that incorporate the novel materials.

It is another object to provide methods of making the foregoing that reduce the use of organic solvent, and also result in improved antimicrobial properties.

### SUMMARY OF THE INVENTION

### Discoveries

We have discovered certain amine-polymer compositions, certain methods of making those compositions, and certain amine-solvent combinations used in making those compositions.

### Amine-Polymer Compositions (AG Polymers)

We have discovered novel amine-polymer compositions having an inhomogeneous concentration of amine therein.

We have discovered a subset of these novel amine-polymer compositions in which, when the composition is immersed in a solution at 37 Celsius (C) having a potential for Hydrogen, (pH) (defined as −log hydrogen ion concentration) in a range around neutral pH:
(1) the amine therein has no detectable (or negligible) leaching from the composition into the solution;
(2) the presence of the composition inhibits growth of certain microorganisms at the interface of the amine-polymer composition and the solution;
(3) the presence of the composition does not inhibit viability of Human Dermal Fibroblasts (HDF) cells in the solution.

For significantly lower solution pH values, these compositions do exhibit detectable leaching of amine into the solution.

### Certain Amines

The certain amines comprise one or more amines selected from the group of primary, secondary, and tertiary amines comprising three R groups. One R group comprises a hydrocarbon group of 6 to 20 carbons. Each one of the two additional R groups comprises one of: a hydrogen and a hydrocarbon group consisting of 1 to 8 carbons.

### Certain Polymers

The certain polymers comprise one or more polymers selected from the group comprising: silicones, polyurethane, other elastomers, polyolefins, polyamides, polyesters, polyethers, and blends of any one or more of the foregoing.

### Methods of Making Amine-Polymer Compositions

We discovered a method of making the novel amine-polymer compositions comprises exposing a surface of polymer to a low Weight Percent (wt %) of amine in an aqueous solution, in which the aqueous solution comprises either an alcohol, DeIonized (DI) water, or a mixture of alcohol and DI water, preferably at temperatures below 100 Celsius (C).

### Products

Any surface of the polymers noted above can be treated with the method to make AG Polymers. Consequently, products include any polymer having a surface treated with the disclosed method. Those products include products having surfaces not intended for contact by humans, products intended for contact with human skin, and products intended for implantation into animal and human bodies.

Products not intended for contact by humans include air filter media, air ducts, and air vents.

Products intended for contact to human skin include clothing, counters, conveyor belts, tables, desks, chairs, masks, tables, doorknobs, railings, elevator buttons, and ear plugs.

Products intended for implantation into animals or humans include implantable tubes, catheters, drainage bags, and prosthetic implants including shunts, stents, and meshes. These implantable products preferably have the very low levels of leaching disclosed herein, in addition to the antimicrobial action and the human biocompatibility also shown herein.

The use of the implantable products comprise the step of implanting at least part of the product into a human body. Foley catheters may be implanted into the bladder through the urethra. Shunts may be implanted in the brain to drain excess cerebrospinal fluid.

Further, the novel AG Polymers have an areal concentration of amine that is so low that implantable medical devices treated by our method would result in products that have a total amine quantity that is substantially less than the amine LD50.

We provide novel compositions comprising:
a quantity of polymer;
a quantity of amine;
wherein the quantity of polymer comprises a first number of monomer units;
wherein a number of (Si(CH3)2O) monomer units in the quantity of polymer is less than 99 percent of the first number of monomer units;
wherein the quantity of polymer comprises one or more polymers selected from the group consisting of silicones, polyurethanes, other elastomers, polyolefins, polyamides, polyesters, polyethers, and blends and copolymers of any one or more of the foregoing;
wherein the quantity of amine consists one or more amines, and combinations thereof;
wherein each one of the one or more amines is selected from the group of primary, secondary, and tertiary amines comprising three R groups;
wherein, for each one of the one or more amines selected from the group of primary, secondary, and tertiary amines,
(1) a first one of the three R groups is a hydrocarbon group of 6 to 20 carbons,
(2) a second one of the three R groups is a member selected from the group consisting of a hydrogen and a hydrocarbon group consisting of 1 to 8 carbons, and
(3) a third one of the three R groups is a member selected from the group consisting of a hydrogen and a hydrocarbon group consisting of 1 to 8 carbons;
wherein the composition occupies a composition volume delimited by a composition surface defining a composition surface area;
wherein the quantity of amine has a weight between 0.001 and 10 milligrams per square centimeter times the composition surface area; and
wherein the quantity of amine is inhomogeneously distributed in the composition volume.

We also provide novel compositions comprising a quantity of polymer and a quantity of amine, formed by a water-rich process comprising:
preparing a mixture comprising (1) an aqueous solution comprising more than fifty percent by volume of water and (2) amine comprising the quantity of amine; and
exposing the quantity of polymer to the mixture;
wherein the quantity of polymer comprises one or more polymers selected from the group consisting of silicones, polyurethanes, other elastomers, polyolefins, polyamides, polyesters, polyethers, and blends and copolymers of any one or more of the foregoing;
wherein the quantity of amine consists of one or more amines, and combinations thereof;
wherein each one of the one or more amines is selected from the group of primary, secondary, and tertiary amines comprising three R groups;
wherein, for each one of the one or more amines selected from the group of primary, secondary, and tertiary amines,
(1) a first one of the three R groups is a hydrocarbon group of 6 to 20 carbons,
(2) a second one of the three R groups is a member selected from the group consisting of a hydrogen and a hydrocarbon group consisting of 1 to 8 carbons, and
(3) a third one of the three R groups is a member selected from the group consisting of a hydrogen and a hydrocarbon group consisting of 1 to 8 carbons;
wherein the composition occupies a composition volume delimited by a composition surface defining a composition surface area;
wherein the quantity of amine has a weight between 0.001 and 10 milligrams per square centimeter times the composition surface area; and
wherein the quantity of amine is inhomogeneously distributed in the composition volume.

We also provide novel compositions comprising a quantity of polymer and a quantity of amine, formed by a process comprising:
preparing a mixture comprising a solution and amine comprising the quantity of amine, and wherein the solution comprises at least one of water and alcohol;
exposing the quantity of polymer to the mixture;
wherein the quantity of polymer comprises a first number of monomer units;
wherein a number of (Si(CH3)2O) monomer units in the quantity of polymer is less than 99 percent of the first number of monomer units;
wherein the quantity of polymer comprises one or more polymers selected from the group consisting of silicones, polyurethanes, other elastomers, polyolefins, polyamides, polyesters, polyethers, and blends and copolymers of any one or more of the foregoing;
wherein the quantity of amine consists one or more amines, and combinations thereof;
wherein each one of the one or more amines is selected from the group of primary, secondary, and tertiary amines comprising three R groups;
wherein, for each one of the one or more amines selected from the group of primary, secondary, and tertiary amines,
(1) a first one of the three R groups is a hydrocarbon group of 6 to 20 carbons,
(2) a second one of the three R groups is a member selected from the group consisting of a hydrogen and a hydrocarbon group consisting of 1 to 8 carbons, and
(3) a third one of the three R groups is a member selected from the group consisting of a hydrogen and a hydrocarbon group consisting of 1 to 8 carbons;
wherein the composition occupies a composition volume delimited by a composition surface defining a composition surface area;
wherein the quantity of amine has a weight between 0.001 and 10 milligrams per square centimeter times the composition surface area; and
wherein the quantity of amine is inhomogeneously distributed in the composition volume.

We also provide novel methods of making a composition comprising a quantity of polymer and a quantity of amine, comprising:
preparing a mixture comprising a water-rich aqueous solution comprising more than fifty percent by volume of water, and amine comprising the quantity of amine; and
exposing the quantity of polymer to the mixture;
wherein the quantity of polymer comprises one or more polymers selected from the group consisting of silicones, polyurethanes, other elastomers, polyolefins, polyamides, polyesters, polyethers, and blends and copolymers of any one or more of the foregoing;
wherein the quantity of amine consists one or more amines, and combinations thereof;
wherein each one of the one or more amines is selected from the group of primary, secondary, and tertiary amines comprising three R groups;
wherein, for each one of the one or more amines selected from the group of primary, secondary, and tertiary amines,
(1) a first one of the three R groups is a hydrocarbon group of 6 to 20 carbons,
(2) a second one of the three R groups is a member selected from the group consisting of a hydrogen and a hydrocarbon group consisting of 1 to 8 carbons, and
(3) a third one of the three R groups is a member selected from the group consisting of a hydrogen and a hydrocarbon group consisting of 1 to 8 carbons;
wherein the composition occupies a composition volume delimited by a composition surface defining a composition surface area;
wherein the quantity of amine has a weight between 0.001 and 10 milligrams per square centimeter times the composition surface area; and
wherein the quantity of amine is inhomogeneously distributed in the composition volume.

We also provide novel methods of making a composition comprising a quantity of polymer and a quantity of amine, comprising:
preparing a mixture comprising a solution and amine comprising the quantity of amine, wherein the solution comprises at least one of water and alcohol; and
exposing the quantity of polymer to the mixture;
wherein the quantity of polymer comprises a first number of monomer units;
wherein a number of (Si(CH3)2O) monomer units in the quantity of polymer is less than 99 percent of the first number of monomer units;
wherein the quantity of polymer comprises one or more polymers selected from the group consisting of silicones, polyurethanes, other elastomers, polyolefins, polyamides, polyesters, polyethers, and blends and copolymers of any one or more of the foregoing;
wherein the quantity of amine consists one or more amines, and combinations thereof;
wherein each one of the one or more amines is selected from the group of primary, secondary, and tertiary amines comprising three R groups;
wherein, for each one of the one or more amines selected from the group of primary, secondary, and tertiary amines,
(1) a first one of the three R groups is a hydrocarbon group of 6 to 20 carbons,
(2) a second one of the three R groups is a member selected from the group consisting of a hydrogen and a hydrocarbon group consisting of 1 to 8 carbons, and
(3) a third one of the three R groups is a member selected from the group consisting of a hydrogen and a hydrocarbon group consisting of 1 to 8 carbons;
wherein the composition occupies a composition volume delimited by a composition surface defining a composition surface area;
wherein the quantity of amine has a weight between 0.001 and 10 milligrams per square centimeter times the composition surface area; and
wherein the quantity of amine is inhomogeneously distributed in the composition volume.

Each one of the foregoing novel compositions and methods of making these compositions may include any one or more of the following additional structures, steps, limitations, or include the noted resulting properties:

The quantity of amine has a weight between 0.032 to 1.280 milligrams per square centimeter times the composition surface area.

The quantity of amine comprises an amine having the first one of the three R groups being a hydrocarbon group of 10 to 17 carbons.

The quantity of polymer comprises a thermoset.

The quantity of polymer comprises one or more polymers selected from the group consisting of polyurethanes, polyolefins, polyamides, polyethers, elastomers other than polydimethylsiloxane (PDMS), and blends and copolymers of any one or more of the foregoing.

The quantity of amine comprises an amine having the first one of the three R groups being an alkyl group of 6-17 carbons.

The quantity of amine comprises an amine that is a secondary amine or a tertiary amine.

The quantity of amine comprises an amine that is a tertiary amine.

The quantity of amine comprises an amine having the first one of the three R groups being a hydrocarbon group of 12 to 15 carbons.

The quantity of amine comprises an amine having the first one of the three R groups being a hydrocarbon group of 14 carbons.

The quantity of amine comprises an amine having the second one of the three R groups being a methyl group.

The quantity of amine comprises an amine having the second and third ones of the three R groups being methyl groups.

The quantity of amine comprises N,N-dimethyltetradecylamine.

The quantity of polymer comprises a silicone.

The quantity of polymer comprises a polyurethane.

The quantity of polymer comprises a polypropylenes.

The quantity of polymer comprises an elastomer.

The quantity of polymer comprises a polyolefin.

The quantity of polymer comprises a polyamide.

The quantity of polymer comprises a polyether.

The quantity of polymer comprises a polyester.

The weight percent of the quantity of amine to the quantity of polymer is between 0.046 to 1.84.

The composition surface area defines a first composition surface area and a second composition surface area;
wherein the first composition surface area opposes the second composition surface area;
wherein a upper region defines an upper region volume extending between the first composition surface area and the second composition surface area to an upper region bottom;
wherein the upper region has an upper region amine density defined as weight of the quantity of amine in the upper region volume;
wherein a lower region defines an lower region volume extending between the first composition surface area and the second composition surface area from the upper region bottom to the lower region bottom;
wherein the lower region has an lower region amine density defined as weight of the quantity of amine in the lower region volume; and
wherein the upper region amine density is greater than the lower region amine density.

The composition surface area defines a first composition surface area and a second composition surface area;
wherein the first composition surface area opposes the second composition surface area;
wherein a upper region defines an upper region volume extending between the first composition surface area and the second composition surface area to an upper region bottom;
wherein the upper region has an upper region amine density defined as weight of the quantity of amine in the upper region volume;
wherein a lower region defines an lower region volume extending between the first composition surface area and the second composition surface area from the upper region bottom to the lower region bottom;
wherein the lower region has an lower region amine density defined as weight of the quantity of amine in the lower region volume; and
wherein an even lower region defines an even lower region volume extending between the first composition surface area and the second composition surface area from the lower region bottom to an even lower region bottom;
wherein the even lower region has an even lower region amine density defined as weight of the quantity of amine in the even lower region volume;
wherein the upper region amine density is greater than the lower region amine density; and
wherein the even lower region amine density is greater than the lower region amine density.

The composition surface includes a first surface region, and wherein concentration of the quantity of amine in the first surface region of the composition surface is inhomogeneous.

The quantity of amine comprises amine molecules of a first amine;
wherein amine molecules of a first amine have a longest dimension; and
wherein orientation of the longest dimension of molecules of the first amine in the composition surface is inhomogeneous.

The quantity of amine comprises amine molecules of a first amine;
wherein amine molecules of the first amine have a longest dimension; and
wherein more of the amine molecules of the first amine in a first surface region of the composition surface have their longest dimension closer to perpendicular, than to parallel, to the first surface region of the composition surface.

The quantity of amine comprises amine molecules of a first amine;
wherein the amine molecules of the first amine have a longest dimension; and
more of the amine molecules of the first amine in a first surface region of the composition surface have their longest dimension close to parallel, than to perpendicular, to the first surface region of the composition surface.

The composition comprises a region having a planer shape.

The composition comprises a region having tubular shape.

The composition comprises a region having a fiber shape.

The composition comprises a region having a porous shape.

The composition comprises a region having a sheet shape.

The composition comprises a region having a bead or particle shape having a dimension from 5 nanometers to 10 millimeters.

The composition further comprises a filler of at least 5% by weight.

The quantity of amine is neither covalently nor ionically bonded to the quantity of polymer.
When the composition is immersed in LB medium having pH of 7.0 plus or minus 0.2, at 37 degrees Celsius for 24 hours, leaching of C14 from the composition into the LB medium is less than 0.042 ug/mL, per square millimeter of the composition surface area.

When a sample having composition surface having a surface area of 239 square millimeters is immersed in 0.6 mL solution of deionized water and KOH having a pH of 7.0, held at 37 C and gently shaken for 1 day, leaching of amine from the sample into the solution is less than 5 ug/mL.

When a sample having composition surface having a surface area of 239 square millimeters is immersed in 0.6 mL solution of deionized water and HCL having a pH of 6.03, held at 37 C and gently shaken for 1 day, leaching of amine from the sample into the solution is less than 5 ug/mL.

When a sample having composition surface having a surface area of 239 square millimeters is immersed in 0.6 mL solution of deionized water and HCL having a pH of 5.53, held at 37 C and gently shaken for 1 day, leaching of amine from the sample into the solution is greater than 5 ug/mL.

The composition provides in antimicrobial efficacy against uropathogenic E. coli and E. faecalis.

The composition provides Human Dermal Fibroblasts (HDF) cell viability greater than 70% as measured based upon ISO standard 10993-5 extraction test.

The composition formed by any of the processes, and process forming the compositions, in which the process further comprises forming the mixture into an emulsion of the quantity of amine in the aqueous solution prior to exposing the quantity of polymer to the mixture.

The number of (Si(CH3)2O) monomer units in the quantity of polymer is less than 90 percent of the first number of monomer units.

The number of (Si(CH3)2O) monomer units in the quantity of polymer is less than 80 percent of the first number of monomer units.

The number of (Si(CH3)2O) monomer units in the quantity of polymer is less than 70 percent of the first number of monomer units.

The composition, per se, the composition as formed by any of the processes, and process forming the compositions, per se, in which the quantity of polymer comprises one or more polymers selected from the group consisting of polypropylene, polyethylene, silicone, polyurethane, nylon, latex, and blends and copolymers of any one or more of the foregoing.

The composition, per se, the composition as formed by any of the processes, and process forming the compositions, per se, comprising forming the mixture into an emulsion of the quantity of amine in the aqueous solution prior to exposing the quantity of polymer to the mixture.

The composition as formed by any of the processes, and process forming the compositions, per se, wherein the water-rich aqueous solution is more than seventy percent by volume of water.

The composition as formed by any of the processes, and process forming the compositions, per se, wherein the water-rich aqueous solution is 100 percent by volume water.

We also provide novel water-rich, amine-poor, emulsion comprising:
an water-rich aqueous solution comprising greater than 50 percent by volume water;
a quantity of amine; and
wherein the quantity of amine is an amount between 0.05 and 0.4 wt % of the aqueous solution;
wherein the quantity of amine consists of one or more amines, and combinations thereof;
wherein each one of the one or more amines is selected from the group of primary, secondary, and tertiary amines comprising three R groups;
wherein, for each one of the one or more amines selected from the group of primary, secondary, and tertiary amines,
(1) a first one of the three R groups is a hydrocarbon group of 10 to 20 carbons,
(2) a second one of the three R groups is a member selected from the group consisting of a hydrogen and a hydrocarbon group consisting of 1 to 8 carbons, and
(3) a third one of the three R groups is a member selected from the group consisting of a hydrogen and a hydrocarbon group consisting of 1 to 8 carbons.

We also provide novel method of making a water-rich, amine-poor an emulsion comprising:
preparing a mixture comprising an aqueous solution comprising greater than 50 percent by volume water, and a quantity of amine; and
mixing the mixture to form the emulsion;
wherein the quantity of amine is an amount between 0.05 and 0.4 wt % of the aqueous solution;
wherein the quantity of amine consists of one or more amines, and combinations thereof;
wherein each one of the one or more amines is selected from the group of primary, secondary, and tertiary amines comprising three R groups;
wherein, for each one of the one or more amines selected from the group of primary, secondary, and tertiary amines,
(1) a first one of the three R groups is a hydrocarbon group of 10 to 20 carbons,
(2) a second one of the three R groups is a member selected from the group consisting of a hydrogen and a hydrocarbon group consisting of 1 to 8 carbons, and
(3) a third one of the three R groups is a member selected from the group consisting of a hydrogen and a hydrocarbon group consisting of 1 to 8 carbons.

The foregoing novel emulsion and method of making an emulsion may include the following additional steps and limitations.

The aqueous solution comprises greater than 70 percent by volume water, greater than 90 percent by volume water, or is 100 percent by volume water.

Forming the mixture into an emulsion of the quantity of amine in the aqueous solution prior to exposing the quantity of polymer to the mixture.

The quantity of amine comprises an amine having the first one of the three R groups being a hydrocarbon group of 10 to 17 carbons.

The quantity of amine comprises an amine having the first one of the three R groups being an alkyl group of 6-17 carbons.

The quantity of amine comprises an amine that is a secondary amine or a tertiary amine.

The quantity of amine comprises an amine that is a tertiary amine.

The quantity of amine comprises an amine having the first one of the three R groups being a hydrocarbon group of 12 to 15 carbons.

The quantity of amine comprises an amine having the first one of the three R groups being a hydrocarbon group of 14 carbons.

The quantity of amine comprises an amine having the second one of the three R groups being a methyl group.

The quantity of amine comprises an amine having the second and third ones of the three R groups being methyl groups.

The quantity of amine comprises N,N-dimethyltetradecylamine.

### DETAILED DESCRIPTION

### Brief Description of the Drawings

FIG. 1 is a side sectional view of composition 1.

FIG. 2 is a top plan view of composition 1.

FIG. 3 is another side sectional view of composition 2.

FIG. 4 is a top plan view of first surface region 400 of a composition.

FIG. 5 is top plan view first surface region 500 of a composition.

FIG. 6 is a collage showing composition shapes 600 to 650.

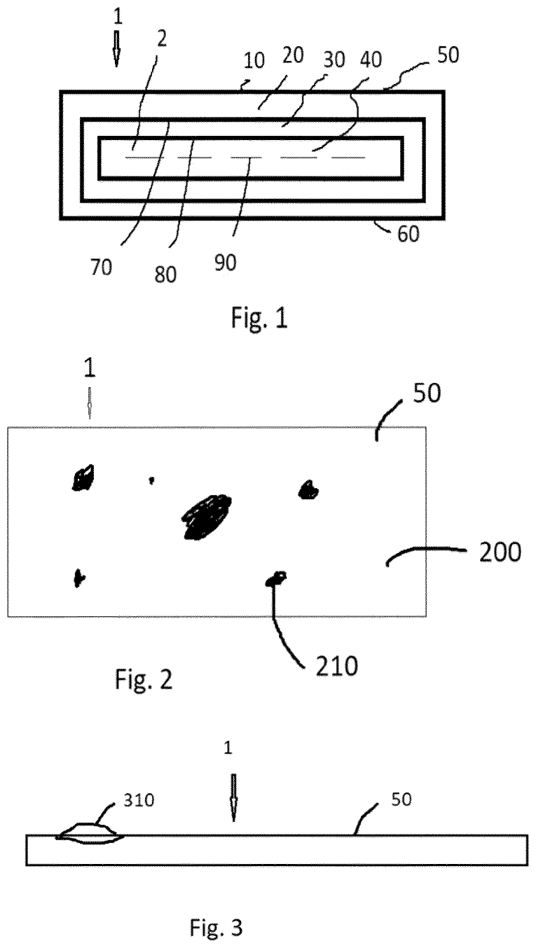

FIG. 1 shows side sectional view of composition 1 including composition surface area 10, upper region 20, lower region 30, even lower region 40, first composition surface area, second composition surface area 60, upper region bottom 70, lower region bottom 80, and even lower region bottom 90. Upper region 20 extends from composition surface area 10 to upper region bottom 70. Lower region 30 extends from upper region bottom 70 to lower region bottom 80. Even lower region bottom 40 extends from lower region bottom 80 to even lower region bottom 90. Even lower region bottom 90 is indicated by a dashed line. Lower region bottom 90 need not be equidistant from first composition surface area 50 and second composition surface area 60, as shown.

FIG. 2 shows region of amine deposition 200 on first composition surface area 50. As shown, amine deposition 200 and other amine deposits are inhomogeneously distributed within region of polymer surface 210.

FIG. 3 shows first composition surface area 50 of composition 2 and region of amine deposition 310 extending from first composition surface area into a composition.

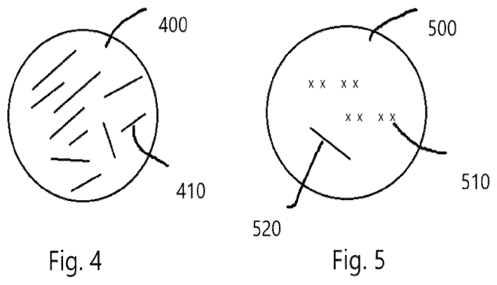

FIG. 4 shows first surface region and amine molecule 410. As shown, amine molecule 410 has an elongated shape with a relatively long dimension. FIG. 4 shows amine molecules including amine molecule 410 are inhomogeneously oriented in first surface region, with more amine molecules oriented in one direction.

FIG. 5 shows first surface region 500, amine molecules 510 of a first amine oriented with their longest dimension close to perpendicular to first surface region 520, and amine molecules of the first amine oriented with their longest dimension close to parallel to first surface region 500.

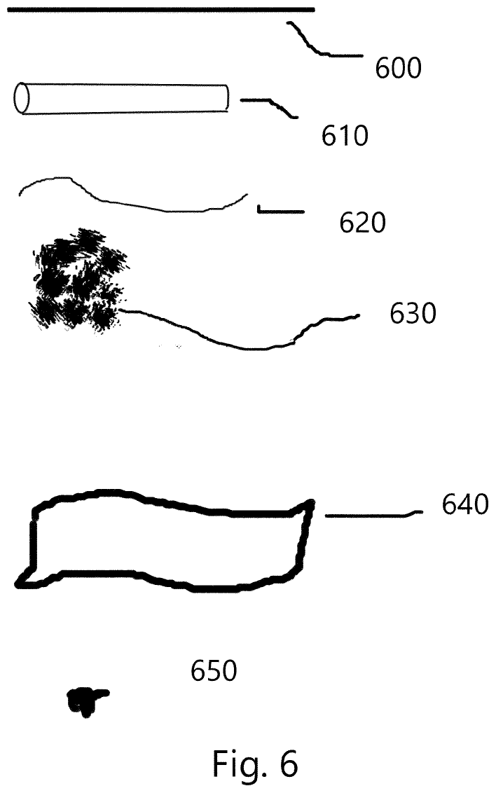

FIG. 6 shows composition 600 having a planar shape, composition 610 having a tubular shape, composition 620 having a fiber shape, composition 630 having a porous shape, composition 640 having a sheet shape, and composition 650 having a bead or particle shape.

### Scope of Amine-Polymer Compositions

We present herein data showing that AG Polymers across the wide range of polymers identified above have antimicrobial properties.

Amines are generally known to have some solubility into the certain polymers identified above. We know that the degree of solubility varies depending upon the particular amine and the composition of polymer. Even for polyamides, we know that there is some degree of solubility. We know that increasing temperature generally increases amine solubility. We know that, the more amorphous a polymer, generally the higher the solubility of the amines mentioned above. In other words, we know that, generally, amines have some solubility across the broad class of certain polymers identified above.

We have discovered that these novel amine-polymer compositions are generally effective at inhibiting growth of microorganisms at normal pH and human body temperature. We discovered that when a surface of amine-polymer compositions contacts certain aqueous solutions containing microorganisms:
(1) the presence of the amine-polymer composition inhibits growth of microorganism;
(2) amine from the amine-polymer composition does not result in appreciable amount of unbound amine molecules in the aqueous solution; and
(3) the antimicrobial response remains present after repeated exposures to microbe containing solutions.

Some of our experiments leading to these discoveries include NMR measurements indicative of the amount of amine extracted from an amine-polymer composition by a methanol extraction, standard antimicrobial activity tests results, surface contact angle measurements of amine-polymer compositions, and visual inspections indicative of the presence or absence of microbes in solutions.

Our methanol extraction tests show that certain polymer-amine composition samples retain substantial amounts of amine after one or more than one methanol extractions.

Our microbial activity tests showed that samples retained antimicrobial activity after repeatedly exposing the sample to fresh solutions containing a known concentration of microorganisms. This indicates that the organism is not binding to the surface of the polymer-amine composition.

Our Immersion Growth (IG), GC-MS, sequential shake flask, and dialysis bag tests show that these polymer-amine compositions do not leach significant quantities of C14 at normal pH and human body temperature. These tests also indicate that the amine bound to portions of the microorganism is not retained at the amine-polymer composition's surface. That is, there is limited or negligible biofouling.

Amine-Polymer Compositions Resulting from Contacting Polymer to Amine in Aqueous Solutions Containing High Concentrations of Water

We have discovered that contacting polymers to combinations of amine in an aqueous solution comprising a relatively high concentration of water and low concentration of alcohol forms a different composition than contacting the same polymer to combinations of amine in an aqueous solution comprising a relatively low concentration of water and high concentration of alcohol.

We discovered that the Contact Angles (CA) of DI water on amine-polymer compositions formed by polymer contact to aqueous solutions comprising a low composition of water are different from those compositions formed by contact with aqueous solutions comprising a high concentration of water.

We have discovered that the combinations of certain amines, such as the C14 amine and aqueous solution comprising a relatively high concentration of water and low concentration of alcohol, can form an emulsion that is sufficiently stable to be used in forming novel amine-polymer compositions.

We have discovered that a relatively lower weight percent of amine in aqueous solutions having relatively high concentration of water can produce compositions having the same areal concentration of amine as a relatively higher weight percent of amine in aqueous solutions having relatively high concentration of alcohol, upon application of a polymer to the same time and exposure to the solutions. That is, we have discovered that the high water concentration solutions results in amine accumulation in the composition at a significantly faster rate than the low water concentration alcoholic solutions.

And we have found that the compositions resulting from use of the high water concentration solutions provide superior inhibition of growth of certain microorganisms than compositions resulting from exposure to aqueous solutions having a relatively low concentration of water.

### Limits of Emulsion Formation by Concentration of Water in Solution

At room temperature, we formed aqueous solutions of various volume ratios of DI water and ethanol to which we added 0.2 percent, by weight, of C14 amine. We found that the C14 completely dissolved in mixtures having up to 45 percent (%) by volume water. We found that the C14 did not completely dissolve and formed an emulsion for aqueous volume fractions of water from 50 to 100% by volume water.

Preferably, the water forms greater than 50%, preferably greater than 70 percent, even more preferably greater than 90 percent, and in the preferred embodiment all of the aqueous solution, other than the included amine.

### Limits of Emulsion Formation by Amine Molecule Size

At room temperature, we added 1.2 wt % of C6 primary alkylamine, into DI water. This amine at this concentration entirely dissolved.

Aniline and other amines containing aromatic groups are known to be more soluble than alkyl (acyclic) amines.

For primary amines, at 0.2 wt. % in DI water at 40 C, we believe (without testing) that hydrocarbon groups greater than C9 (C10 to C20) can form emulsions and that hydrocarbon groups of C9 and below will not form emulsions.

For secondary and tertiary amines lacking aromatic groups, we believe that hydrocarbon groups greater than C9 (C10 to C20) can form emulsions.

### Limits of Emulsion by Concentration of Emulsifiers

A higher concentration of amine may be emulsified into solution by addition of an emulsifier. However, emulsifiers also hinder amine uptake into the composition.

Preferably, the molecular ratio of any emulsifier to amine in the mixture is less than 1:5, more preferably less than 1:50, and preferably 0.

### Amine Compositions

The amines in the composition may comprise more than one species of the group of C6-C20(NR1R2) where R1 and R2 refer to the two additional R groups (that include H) mentioned above.

The amines in the composition may comprise a mixture of at least two amines each having one hydrocarbon group with 12, 14, 16, 18, and 20 carbon atoms.

The amines in the composition may comprise one of, or mixture of at least two amines, having carbon chains that have one or more of 6, 8, 10, 12, 14, 16, 18, and up to 20 carbon atoms.

The amines in the composition may comprise a mixture of at least two amines, each having a hydrocarbon group that has 12, 14, 16, 18, or 20 carbon atoms.

The amines in the composition may comprise one of, or mixture of at least two amines, each having a hydrocarbon group that has 12 or 14 carbon atoms.

The amines in the composition may comprise primary amines.

The amines in the composition may comprise secondary amines.

The amines in the composition may comprise tertiary amines.

Preferably, the amines in the composition comprise an amine having a carbon chain having 14 carbon atoms.

The amines in the composition may comprise one species Cx(NR1R2) and one species Cy(NR1R2) of the group of C6-C20(NR1R2), in which x is not equal to y. And x may in this case be 12, 14, or 16, and y may be 12, 14, or 16.

The amines in the composition may comprise one species of the group of C6-C20(NRxRy) selected from the group C6-C20(NR1R2), in which x is not equal to y. And in this case x may be a methyl group, and y may be an ethyl group.

Preferably, the composition comprises the amine N,N-dimethyltetradecylamine (CH3(CH2)13N(CH3)2, CAS No. 112-75-4).

### Polymer Compositions

The polymers may include cross-linkers and fillers such as SiO2, as is well known in the polymer arts.

The polymers include polymers that are thermosets, and polymers that are not thermosets, that is thermoplastics.

The silicones comprise polysiloxanes. The polysiloxanes may comprise polydiorganosiloxanes. The polydiorganosiloxanes may comprise one or more of the following: (1) polydimethylsiloxane, (2) copolymers comprising polydimethylsiloxane, (3) copolymers comprising dimethylsiloxane moieties and diphenylsiloxane moieties, and (4) copolymers comprising triflouromethylmethylsiloxane and dimethylsiloxane moieties.

The other elastomers may comprise polybutadiene and latex.

The polyolefins may comprise polyethelyenes and polypropylenes.

The polyamides may comprise Nylon 6 and Nylon 66.

Exclusions from the Aqueous Solution

Preferably, no quaternary amine is included in the aqueous solution.

Preferably, no amine salt is included in the aqueous solution.

Preferably, no amine including aromatic groups or cyclic groups is included in the aqueous solution.

Preferably, no precipitant for an amine in the aqueous solution is included. For example, oxalic acid and salts thereof are precipitants for C14 in aqueous solution at 40 C, and should not be included when C14 in aqueous solution at 40 C, since that will inhibit or prevent formation of the AG polymer.

Preferably, no strong acid, alkyl halide, or acetate is included in the aqueous solution with the amine, since they react with and/or precipitate amines.

Preferably, no aldehyde or ketone is included in the aqueous solution, in case the amine is a primary or secondary amine, since those react with the same.

### Amine-Polymer Properties

Preferably, the amine is not covalently bonded to either the polymer or polymer surface, either directly or through a linker.

The amines may be in contact with a subsurface region of the polymer, the surface of the polymer, or both.

The concentration of amine per surface area for an amine-polymer composition may be controlled by time and temperature of exposure of the polymer to the amine.

For AG Polymers, specifically including AG Silicone, forming by submerging the polymer in a mixture of amine, such as C14, in aqueous solution at 40 C for 1 hour results in an AG Polymer in which a substantial fraction of the amine is diffused below surface of the polymer, as opposed to residing above the surface of the polymer. This fraction may be greater than 10% of the amine, greater than 50% of the amine, or greater than 90% of the amine.

The amine may be C14, and may have an areal density on the polymer of 1.9×10 to the power of 18, of C14 molecules, per square centimeter. This areal density is equivalent to 80 molecular layers of C14 on the polymer.

The amine in the composition may have a weight per polymer surface area that is between 10 and 100 times the weight per polymer surface area of a monolayer of the amine having their C6-C20 chains extending vertically away from the surface.

The amine in the composition may have a weight per polymer surface area that is between 10 and 5000 times the weight per polymer surface area of a monolayer of the amine having their C6-C20 chains extending along the surface.

The amine in the composition may have a C12 chain, and the amine weight per surface area may be between 900 and 1100 times the weight per polymer surface area of a monolayer of the amine having their C6-C20 extending along surface.

The amine may have a C12 chain, and the amine weight per surface area may be between 17 and 76 times the weight/area of a monolayer of the amine having their C12 chain extending along surface.

Preferably, the concentration of amine per surface area is between 0.01 and 2.00 milligrams per square centimeter (mg/cm2), more preferably between 0.1 to 0.9 mg/cm2, and the currently preferred value is 0.2 mg/cm2.

The concentration of amine per amine-polymer composition surface area may be in any range having endpoints of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, and 2 mg/cm2.

Preferably, the composition is stable under human body temperature in air and in water at pH 7 and at pH 7.4. Preferably, the composition is stable in physiological fluid, blood, saliva, mucosa, plasma, cerebrospinal fluid, and nasolacrimal fluid. Preferably, the composition is stable in urine having a pH greater than 6.03. In this paragraph, stable, means that the log reduction value for E. coli ATCC 700928 samples using the ASTM-2149 “shake flask” test for antimicrobial effectiveness decreases by no more than 30 percent after 1 week.

Preferably, the composition is antimicrobial and cytocompatible.

Preferably, the composition is antimicrobial to at least one of Gm+ bacteria, Gm− bacteria, and yeast Candida albicans.

### Methods of Making AG Polymers, and Novel Mixtures of Amines and Solution

We discovered a method of making the novel AG Polymers which comprises exposing a surface of polymer to a low weight percent of amine in an aqueous solution, in which the aqueous solution comprises either an alcohol, DI water, or a mixture of alcohol and DI water, at temperatures below 100 Celsius (C).

Time, temperature, and starting concentration of amine in aqueous solution can be chosen to result in a desired quantity of amine in the resulting AG polymer, either as measured by areal concentration or by wt % (weight of amine to weight of substrate).

A preferred concentration range for amine in aqueous solution is between 0.01 and 3 wt %, more preferably between 0.05 and 0.4 wt %, and more preferably between 0.1 and 0.3 wt %. The currently preferred concentration is 0.2 wt %.

Preferably, the temperature is less than 100 C and greater than 0 C, more preferably less than 80 C and greater than 10 C.

Immersion time preferably is between 1 minute and 8 hours, more preferably between 10 minutes and 2 hours.

The currently preferred time, temperature, and amine concentrations are 0.2 wt %, 40 C, and 1 hour.

Preferably, the AG polymer samples are washed in DI water upon removal from the aqueous solution.

Preferably, the aqueous solution has a sufficiently high concentration of DI water for the chosen amine, so that the amine is not entirely solvated, so the mixture of aqueous solution and amine can be emulsified. We have found that aqueous solutions having high water concentrations in which the amine is not entirely solvated can be emulsified. And we have found that AG polymers formed by submerging polymer in the high water concentration aqueous solutions form different compositions than formation by submerging the polymer in aqueous solutions lower in water concentration and higher in alcohol concentration.

These AG polymers differ in antimicrobial activity, contact angles, and amine distribution within the AG Polymers. To distinguish based upon their properties, we refer herein below to AG water-based polymers and AG alcohol-based polymers. The use of water-based and alcohol-based refers to the difference in properties, does not require the claimed composition be formed by a water-based or alcohol-based process merely because that phrase appears in the AG Polymer names used below.

### AG Polymers

Compositions comprising polymer and amine are named AG Polymers.

AG Polymers compositions formed using water-rich aqueous solutions are called AG water-based polymers.

AG Polymers compositions formed using alcohol-rich aqueous solutions are called AG alcohol-based polymers.

AG water-based polymers differ from AG alcohol-based polymers, for the same polymer, as determined by advancing, receding, and static contact angles when in contact with DI water, ratio of weight of polymer to weight of amine, and areal concentration of amine.

### Polymer Sources for AG Water-Based and Alcohol-Based Polymers

The source of the untreated polymers used to prepare the AG water-based Polymer samples identified below, were:
Polypropylene: McMaster-Carr 8742K531 Polypropylene (0.0625-inch=0.15875 centimeter thickness sheet, formed into 0.7 cm diameter disks).
Polyethylene: TIVAR 1000 Natural Plate Ultra High Molecular Weight Polyethylene (0.0625-inch=0.15875 centimeter thickness sheet, formed into 0.7 cm diameter disks).
Nylon: TECAMID® 66 natural (0.01-inch=0.0254 centimeter thickness sheet, formed into 0.7 cm diameter disks). TECAMID® 66 specifies Polyamide 66, which is made from condensation of hexamethylenediamine and adipic acid monomers.
Latex: Hygienic Natural Rubber Tubing HPN-10910 (having outer diameter 0.90 cm and inner diameter 0.70 cm. Natural rubber is a polymer comprising cis-1,4-polyisoprene.) Latex samples were formed by cutting Latex pipe segments 0.5 cm long extending along the tube axis of symmetry, and then sliced along the axis of symmetry to form half-pipe segments.
Polyurethane: Lubrizol SG 80A in the form of beads. Only AG water-based Polyurethane samples were prepared from these beads. We made antimicrobial, amine weight percent, and amine areal density determinations from these samples.
Polyurethane: EZ-FLO ⅛-inch (0.3175 centimeter) inner diameter, ¼-inch (0.635 centimeter) outer diameter micro fuel line (that is, Lowe's store item 879284, model 98641. AG water-based and alcohol-based Polyurethane samples prepared using portions of this fuel line were used for contact angle measurements.
Silicone: Bardex All-Silicone Foley Catheter, French 22 (SKU: 165822). Size French 22 has an outer diameter of 0.733 centimeters, inner diameter is 0.533 centimeters, wall thickness 0.1 centimeters.

### Preparation of AG Water-Based Polymer Samples

AG water-based polymers comprising the C14 amine (CAS 112-75-4) were formed by the following steps in the sequence in which they appear.

Placing 0.24 g C14 amine in a flask with 120 mL of DI water to make a 0.2 wt. % C14 in water mixture.

Mixing the 0.2 wt. % C14 in water mixture with a Scilogex d160 homogenizer for 10 min at 30,000 RPM. This mixing resulted in the mixture forming an emulsion.

Separating the 120 mL mixture into 20 mL batches.

Heating while continuously stirring a batch until its temperature reached 40 Celsius (C).

Immersing polymer samples into one of the 40 C batches.

Leaving the polymer samples immersed in the batch for 1 hour (H) while continuing the stirring. This step forms the AG water-based polymer samples.

Washing the AG water-based polymers samples with DI water.

Repeating the step of washing the AG water-based polymers samples with DI water two more times, for a total of three washing steps.

### Preparation of AG Alcohol-Based Polymer Samples

AG alcohol-based polymers comprising C14 amine were formed by the following steps in the sequence in which they appear.

Placing 0.6 g C14 amine (CAS 112-75-4) into flasks containing 19.4 g of ethanol (lab-grade 95% ethanol, denatured with isopropyl alcohol and methanol) to make a mixture of 3 wt. % C14 in alcohol.

Repeating the placing step 6 times to make 6 batches of mixture.

Heating while continuously stirring a batch until its temperature reached 40 C.

Immersing polymer samples into one of the 40 C batches while continuing the stirring.

Leaving the polymer samples immersed in the batch for 1 hour. This step forms the AG alcohol-based polymer samples.

Washing the AG alcohol-based polymers samples with DI water.

Repeating the step of washing the AG alcohol-based polymers samples with DI water two more times, for a total of three washing steps.

### Resulting AG Polymer Samples

Each batch of was used to create AG polymer samples from polypropylene (46 disks), polyethylene (46 disks), polyurethane (12 half-pipe segments), polyurethane (beads having total weight of 3.34 grams), latex (30 segments), nylon (62 disks), silicone (20 half-pipe segments).

### Measurement Procedures for AG Polymer Samples

AG polymer samples intended for antimicrobial testing were placed in phosphate buffer solution, pH 7.2, sterilized by UV exposure for 24-hours, and then placed in the antimicrobial testing solution, and tested for antimicrobial efficacy, using the “shake flask test”, consistent with ASTM E2149, and as described by Wynne et al., and further described herein below.

AG polymer samples intended for contact angle testing were then dried by dabbing with disposable, lint-free wipes. Advancing, receding, and static contact angles of DI water on the samples were measured as described in Wynne et al.

### Amine Weight and Areal Coverage Procedures

The weight of amine incorporated into a set of AG polymer samples formed using the same polymer, was determined using 1H-NMR spectroscopy and deuterated methanol extraction by generating a standard curve, and then comparing to the standard curve.

A standard curve relating weight in milligrams per milliLiter (mL) of C14, to a ratio of integrated 1H-NMR signal for the terminal methyl group on C14 divided by integrated 1H-NMR signal for deuterated methanol containing the C14, was established.

One or more C14 extractions and NMR measurements were performed, as follows. A set of AG polymer samples were placed in a container containing deuterated methanol. The container was closed and maintained at 37 C while gently shaken for 21 plus or minus 4 hours. The same NMR procedure noted above for the standard curve generation, was conducted on a fraction the solution from the container. The quantity of C14 extracted from set of AG polymer samples into the deuterated methanol was calculated based upon the resulting NMR data, the fraction of the solution from the container, and the standard curve.

For each set of AG polymer samples, the C14 extraction was repeated until either the extraction resulted in an amount of C14 deemed to be insignificant to the total amount of C14 extracted or the amount of C14 obtained in the extraction fell below the noise level for detection of C14. For each set of AG Polymer samples, the total quantity of C14 from the extractions for that set were summed to provide a measure of the total quantity of amine in that set of AG Polymer samples.

The weight of polymer in each set of AG polymer samples was determined using an analytical balance.

The surface area of each set of AG polymer samples was determined by length measurements.

### Extractions and Weights for AG Water-Based Polymer Samples

For the AG water-based Nylon samples, a single extraction was performed because that provided an amount of amine near the noise level. For the AG water-based Polypropylene samples, two extractions were performed. For the AG water-based Polyethylene samples, three extractions were performed.

For the AG water-based Polyurethane samples, SG 80A Lubrizol beads, six extractions were performed.

For the AG water-based Latex samples, six extractions were performed.

For the AG water-based silicone, 3 extractions were performed.

The weight of the untreated polymers, from which each set of AG water-based Polymer samples were formed for these extractions, was: Nylon 0.504 gram; Polypropylene 0.578 grams; Polyethylene 0.736 grams; Latex 0.6876 grams; For the bead based Polyurethane samples 0.8355 grams; and Silicone 0.836 grams.

For the AG alcohol-based silicone, 4 extractions were performed, and the silicone weight was 0.836 grams.

In data tables presented herein, “X” indicates lack of data.

### Contact Angle, C14 Weight %, and C14 Areal Coverage Data

Data for untreated polymers, C14 including AG water-based polymers, and AG alcohol-based polymers appear in the following Table 1, in which semicolons delimit data names and values. In Table 1:
U means Untreated, and refers to polymer not treated with an amine combination.
W means Water treatment, and refers to polymer treated with a combination of amine with a high water concentration aqueous solution.
A means Alcohol treatment, and refers to polymer treated with a combination of amine with a high ethanol concentration aqueous solution.
ACA means Advancing Contact Angle. RCA means Receding Contact Angle. SCA means Static Contact Angle.
W % means ratio, in percent, of Weight of amine in the sample to Weight of polymer in the sample.
AC means Areal Concentration of the amine in mg/cm2.
In the tables, semicolons delimit data names and values and “X” indicates lack of data.
Weight, in grams, of polymer samples used to determine wt % and AC are: Nylon: 0.504; Polypropylene: 0.578; Polyethylene: 0.736; Latex: 0.6876; Polyurethane beads: 0.8355.

TABLE 1

| Polymer | ACA(U; W; A) | RCA(U; W; A) | SCA(U; W; A) | W % (W; A) | AC(W; A) |
|---|---|---|---|---|---|
| Polypropylene | 100.5; 87.7; 106.9 | 55.3; 58.2; 19.9 | 101.2; 80.7; 96.1 | 0.305; X; | 0.143; X |
| Polyethylene | 103.9; 91.6; 105.7 | 63.8; 56.0; 12.3 | 98.5; 84.9; 96.1 | 0.324; X; | 0.194; X |
| Silicone | 105.7; 94.8; 105.8 | 70.4; 62.1; 40.6 | 96; 88.4; 97.6 | 0.92; 1.013 | 0.64; 0.71 |
| Polyurethane | 94.6; 85.7; 81.4 | 34.9; 60.6; 27.3 | 85.3; 78.8; 76.7 | 1.188; X; | 0.596; X |
| Nylon | 67.6; 35.2; 41.2 | 13.1; 11.5; 12.0 | 59.1; 30.2; 40.8 | 0.013; X; | 0.005; X |
| Latex | 97.4; 78.3; 103.5 | 0.0; 0.0; 0.0 | 80.4; 67.4; 95.0 | 1.479; X; | 0.848; X |

This table shows that the AG water-based polymers including polypropylene, polyethylene, Latex, Nylon, and Silicone have lower ACAs and SCAs than the corresponding AG alcohol-based polymers.

This data shows that the AG water-based Polyurethane has a slightly higher ACA and SCA than the corresponding AG alcohol-based Polyurethane.

This data shows that the AG water-based polymers including polypropylene, polyethylene, silicone, and polyurethane have higher RCAs than the corresponding AG alcohol-based polymers.

These results show that AG water-based polymer samples are different than the corresponding AG alcohol-based samples.

Nylon shows a relatively low weight percent and areal concentration of C14, indicating AG Nylons should be prepared at higher temperature and/or for longer time to incorporate more C14 amine.

### Implantable Device Amine Quantity Compared to LD50

For C14, acute oral toxicity for rats is 1320 milligrams per kilogram of body weight. Rats are a proxy for human toxicity.

A 22 French Foley catheter tube 46 cm long, and 0.73 cm in diameter has a surface area of 211 square centimeters. The oral toxic dose for a 1 kilogram rat of 1320 milligrams of C14, if distributed over 211 square centimeters, would provide an areal density of 6.2 mg/cm2 of C14. Our Areal Concentration (AC) data, see Table 1, reports areal concentrations no larger than 0.848 mg/cm2.

### Amine Depth Penetration Testing and Results, DI Water Versus Ethanol

Half-pipe segment shaped, 1 centimeter long polymer samples were formed from a commercial silicone catheter (Bard brand), by cutting. The sample was transparent.

Silicone is not permeable to Ninhydrin in DI water. Ninhydrin stains primary amines. Primary amines were used with Ninhydrin to obtain information on amine depth distribution in AG polymers.

### Tetradecylamine (C14 Primary Amine)

A first mixture was formed by adding 0.2 wt % Tetradecylamine to DI water and emulsified in a sonication bath at 60 C.

A second mixture was formed by adding 3.0 wt % Tetradecylamine to ethanol.

A third mixture was formed by adding 2 wt % ninhydrin to DI water.

Silicone samples were placed in the first and second mixtures for one hour at 70 C, forming AG Silicone samples.

Then the samples were removed from their containers, rinsed with DI water, and immersed in separate containers having the third mixture at 70 C for 30 minutes.

Visual inspection of the samples showed they were darkened. That darkening indicates that ninhydrin on the surface of the AG polymer reacted with amine from the AG polymer.

Then the samples were removed from the third mixture and rinsed with ethanol for 30 seconds, and then dried with fiber free wipes. During the ethanol rinsing, no change in the color of the ethanol was observed. That lack of color change indicates minimal amine extraction from the AG polymers during ethanol washing.

Then the samples were cut, exposing a cross-section extending from an inner surface of the half tubular shape to the outer surface of the half tubular shape.

The samples including the portion having the exposed cross-section were placed in the third mixture (ninhydrin), again, at 70 C for 30 minutes.

The sample was then removed from the third mixture.

The AG water-based and AG alcohol-based samples both showed differences in staining as a function of depth from the original surfaces. This means that in both the AG water-based polymer sample and the AG alcohol-based polymer sample, the distribution of amine in the samples is inhomogeneous as a function of depth from the original surfaces.

However, visual examination of staining also indicated that the amine concentration versus depth from the original surfaces differed for the AG water-based polymer sample and the AG alcohol-based polymer sample.

Visual examination of the cross-section of the AG water-based polymer sample shows the original darkening on the original surfaces, and relatively little darkening in the surface defined by the cross-section. This indicates that there was virtually no amine deep within the AG water-based polymer sample.

Visual inspection of the cross-section of the AG alcohol-based polymer sample shows the original darkening on the original outer surfaces of the sample, but it also shows significant darkening throughout the depth between opposing original surfaces, except in a relatively narrow region beneath the original surfaces where there is virtually no darkening. This indicates that amine in this AG alcohol-based polymer sample preferentially distributed into the central region of the bulk, avoiding a thin region of the bulk adjacent to the original surfaces.

Tetradecylamine and C14 are analogs. Therefore, these results for Tetradecylamine also indicate that the distribution of C14 in AG polymers are inhomogeneous and differ between AG water-based and AG alcohol-based polymers.

### Antimicrobial Testing and Results, Shake Flask Tests for AG Water-Based Polymers

Antimicrobial efficacy against uropathogenic E. coli and E. faecalis was determined using a 2-sequence, modified ASTM E2149 standard (shake flask test) for AG water-based polymers.

The AG water-based samples were normalized by matching all the surface area per volume of challenge media to approximately 1.31 square centimeters per mL of liquid.

For each shake flask test, the amount of sample used per test was: Polypropylene 6 disks; Polyethylene 6 disks; Nylon 8 disks; Latex 4 half-pipe segments; Polyurethane 0.836 grams.

In the shake flask test procedure, challenge media were made by inoculating a 0.18 milliMolar (mM) Phosphate Buffered solution (PB) with 10 to the 5th power of Colony Forming Units (CFU) per mL of one of the two specified bacteria. Each AG surface modified polymer or control sample (untreated segments) was added to a separate flask containing 5 mL of PB inoculated with bacteria. Duplicates of control and AG polymer tests were performed.

The samples were shaken in solution for 24 hours at 37 degrees Celsius.

Then, we agar-plated an aliquot of the resulting solution.

Then, the agar plates were incubated for 24 hours at 37 degrees Celsius.

Then, we counted CFUs on each plate.

Then, we calculated CFU per mL of the aliquot.

Then, we took log base 10 of the final CFU concentrations.

Then, we subtracted the final log value for the control uncoated polymer sample, the log value for the AG polymer sample. These number are reported as “Log Reduction” (LR).

These results were designated as the 1st sequence test results.

The samples were then rinsed with DI water, then sterilized under UV light in PBS for 24-hours.

The samples were then subject to the same shake flask test procedure a second time, designated as the 2nd sequence.

The bacteria used were Escherichia coli—ATCC #700928 and Enterococcus faecalis—ATCC #29212.

For the 1st sequence, the AG water-based polymer sample test results are in the following Table 2.

TABLE 2

| AG Polymer; | E. Coli LR (% kill); | | E. faecalis LR (% kill) | |
|---|---|---|---|---|
| Polypropylene; | 2.9 | (99.9%); | 4.6 | (99.998%) |
| Polyethylene; | 3.8 | (99.98%); | 4.5 | (99.997%) |

| | | |
|---|---|---|
| Silicone; | >4; | X |

| | | | | |
|---|---|---|---|---|
| Polyurethane; | 4.6 | (99.998%); | 4.8 | (99.998%) |
| Nylon; | 0.12 | (24%); | 1.6 | (97.2%) |

| | | |
|---|---|---|
| Latex; | 0; | 0 |

A reason why Latex shows LR=0 is that the control, which was a natural Latex, killed all bacteria (due to antimicrobial properties inherent in natural Latex). Therefore, LR=0 for Latex indicates all bacteria in both the control sample and the AG sample were killed.

The AG water-based Nylon samples were the least effective, which correlates with their relatively low C14 wt % and areal density (0.013 wt % and 0.005 AC, see Table 1).

To confirm that AG water-based Nylon samples with more amine were effective antimicrobials, a higher C14 concentration sample was prepared and tested. This sample was prepared by immersing the Nylon in a mixture of 1 wt % C14 in DI water at 70 C for 1 hour, and then washing the resulting AG water-based Nylon sample with DI water. This sample showed a LR for the E. coli of 2.8 (kill of 99.8%).

The 1st sequence results and the additional Nylon test show that all AG water-based polymers were effective at killing bacteria.

For the 2nd sequence, the AG water-based polymer sample test results are in Table 3.

TABLE 3

| AG Polymer; | E. coli LR (% kill); | | E. faecalis LR (% kill) | |
|---|---|---|---|---|
| Polypropylene; | 4.8 | (99.998%); | 4.3 | (99.996%) |
| Polyethylene; | 4.6 | (99.997%); | 4.5 | (99.997%) |

| | | |
|---|---|---|
| Silicone; | X; | X |

| | | | | |
|---|---|---|---|---|
| Polyurethane; | 4.1 | (99.99%); | 4.0 | (99.99%) |
| Nylon; | 0.2 | (40%); | 0.1 | (18%) |

| | | |
|---|---|---|
| Latex; | 0; | 0 |

These results show that the surface of all the AG water-based Polymer samples remained effective at killing bacteria subsequent to the 1st sequence, after a DI water rinse and resterilization. This indicates weak adsorption of bacterial cell remnants to the AG water-based Polymer samples (lack of fouling), and presence of C14 at the surface of the AG water-based Polymer samples (renewal).

AG water-based Silicone samples were also prepared by immersion of silicone in mixtures including various weight percents of C14 in DI water for 1 hour at 40 Celsius with stirring, as shown by the left-hand column in the following Table 4.

The 2nd, 3rd, and 4th columns in this table show the log of difference in bacteria counts from AG water-based Silicone samples and untreated Silicone samples for E. coli, A. baumannii, and S. aureus. The 5th column shows percent of Human Dermal Fibroblasts (HDF) that remained viable, as measured based upon ISO standard 10993-5 extraction test.

TABLE 4

| C14 wt %; | E. coli; | A. baumannii; | S. aureus; | HDF |
|---|---|---|---|---|
| 0.01; | 0; | X; | X; | 100 |
| 0.05; | >4; | 0.5; | 0.24; | 100 |
| 0.1; | >4; | 4.5; | 1.5; | 98 +/− 2 |
| 0.2 | >4; | 5.5; | 2.1; | 94 +/− 2 |
| 0.3; | >4 | X; | X; | 78+/2 |
| 0.4; | >4; | X; | X; | 1 +/− 1 |
| 0.5; | >4; | X; | X; | 0 |

This data shows a range of greater than 0.01 and less than 0.4 of C14 wt % in DI water over which resulting AG water-based Silicone samples have antimicrobial effectiveness and for which HDFs remain viable. Based upon the 0.2 wt % resulting in AG water-based silicone sample having 0.92 wt % C14 and 0.64 mg/cm2 C14, linear extrapolating indicates this range corresponds to 0.05*0.92 to 2*0.92 (wherein * indicates multiplication), or 0.046 to 1.84 wt %, and 0.05*0.64 to 2*0.64, or 0.032 to 1.28 mg/cm2 for both antimicrobial effectiveness and HDF cell viability.

### Antimicrobial Testing Comparing Alcohol-Based and Water-Based AG Polymer Antimicrobial Activity

Wynn et al report shake flask test data for C14 AG alcohol-based silicone formed by immersing the same silicone material into a solution of 3 wt % C14 in 95% ethanol denatured with methanol and isopropanol, under the same 40 C and 1 hour immersion with stirring. Wynne et al report this AG alcohol based silicone polymer had 1.1 wt % C14. That sample also had an areal concentration of C14 of 0.77 mg/cm2. Wynne et al Table 3 shows the S. aureus LR was only 0.9. The AG water-based polymer sample formed from the 0.2 W % in DI water described above has slight lower C14 wt % (0.92) and slightly lower areal concentration (0.64 mg/cm2), see Table 1, but a superior LR for S. aureus of 2.1, see Table 4.

### MIC and MBC for C14 Amine

We determined that C14 solutions below about 75 micrograms per milliLiter (ug/mL) were transparent indicating solubility while concentrations greater than about 125 ug/mL were translucent.

We determined Minimum Inhibitory Concentrations (MICs) and Minimum Bactericidal Concentrations (MBCs) of certain pathogens to C14 mixtures by the standard dilution method. Our results are shown in Table 5, in which concentration of C14 is in units of ug/mL.

TABLE 5

| Pathogen; | MIC; | MBC |
|---|---|---|
| S. epidermidis (Gm+); | 12.5; | 25 |
| S. aureus (Gm+); | 25; | 50 |
| E. faecalis (Gm+); | 6.3; | 12.5 |
| K. pneumoniae (Gm−); | 25; | 50 |
| P. aeruginosa (Gm−); | >100; | >100 |
| E. coli (Gm−) uropathogenic; | 10 | 20 |

### Leaching Testing and Results

We used the more sensitive E. coli (Gm−) uropathogenic; MIC of 10 ug/mL as an indication of leaching to quantify C14 amine leaching in Immersion Growth (IG) tests.

### Immersion Growth (IG) Tests on AG Water-Based Silicone Samples

We used the uropathogenic (Gm−) E. coli for Immersion Growth (IG) tests.

This IG testing used AG water-based silicone samples describe in Table 4, formed from mixtures having C14 wt % of 0.05, 0.1, and 0.2.

The IG protocol was:
(1) AG water-based silicones and untreated silicone segments, were added to separate 50 mL flasks containing Luria-Bertani (LB) medium as an extraction medium (1 segment per 1 mL);
(2) The flasks were shaken for 24 hours at 37° C.;
(3) The segments were removed from their respective flasks;
(4) The media were inoculated with uropathogenic E. coli (UPEC, ATCC 700928, 2000 CFU/mL) followed by 24 hours incubation;
(5) 100 uL aliquots from the respective flasks were removed, serially diluted, spread on agar, incubated for 24 hours at 37° C., and colonies counted.

The IG test showed normal uropathogenic E. coli growth after prior immersion of the AG water-based silicones in growth medium for up to 24 h, and normal growth continued to 1 week, after which the test were stopped. Similar testing on AG alcohol based silicone gave almost identical results.

This result indicates that leaching of C14 from the AG water-based silicone samples, and also AG alcohol-based silicone samples, into the LB medium was below 10 ug/mL for immersion for 24 hours (and for 1 week) at 37° C.

AG water-based silicone sample surface area was determined to be 239 square millimeters. Consequently, C14 leaching per area from AG water-based silicone was less than (10/239)=0.042 ug/mL, per square millimeter, for immersion for 24 hours (and for 1 week) at 37° C.

This result supports the contact kill theory for AG water-based silicone samples.

### AG Alcohol-Based Silicone Immersion Tests and Data

Using the same procedure noted for AG water-based silicone, we found C14 leaching at pH 5.53, prevented growth, at 24 (and 48 and 72) hours for AG Alcohol-based silicone. Consequently, C14 leaching per area from AG alcohol-based silicone was greater than 0.042 ug/ml per square millimeter, for immersion for 24 hours at 37° C.

The same test at pH 6.03 resulted in no detectable leaching, which means leaching of less than 0.042 ug/ml per square millimeter.

The LB medium had a pH 7.0 plus or minus 0.2, for both water-based and alcohol-based IG tests.

### GC-MS Leaching Tests and Data

GC-MS testing was done to detect C14 leaching into ultrapure water, with pH adjusted with HCl and KOH, at pH 5 and pH 7. Individual AG water-based Silicone 1-cm half pipe segments were placed in a 2 mL centrifuge tube containing 0.6 mL of pH-adjusted solution. The samples were placed in an orbital shaker and gently shaken at 125 RPM, 37 C for 1 day. At the end of the testing period, 0.5 mL of the exposed solution was removed from the centrifuge tube and used for GC-MS analysis.

At pH 5, we found detectable leaching. At pH 7, we found no detectable leaching. Our detection limit was 5 ug/mL.

AG Alcohol-based silicone immersion tests and data, and GC-MS leaching tests and data on AG water-based Silicone, indicate that C14 leaching from AG silicone polymers does not have detectable leaching when the surrounding media has pH >6.03 and does have detectable leaching when the surrounding media has pH less than or equal to 5.53.

We believe essentially the same pH ranges for leaching exist for all polyolefins (including polyethylene, polypropylene, and blends and copolymers thereof), because they have similar hydrophobic properties to silicone.

Similarly, we believe pH ranges exist where C14 amine does and does not exhibit substantial leaching from AG polymers comprising polyurethanes, other elastomers, polyamides, polyesters, polyethers, and blends and copolymers of any one or more of the foregoing.

### Dialysis Bag Tests

Our dialysis bag tests showed that samples of polymer-amine composition exposed to certain microbe containing fluids did not result in substantial amount of free amine in the fluid, but did inhibit microorganism growth in the fluid. The dialysis bags are permeable to the amine. Fluid inside the dialysis bag in contact with the polymer-amine composition became clear, indicating inhibition of microorganism growth. Fluid outside the dialysis which was in contact with the dialysis bag was or became cloudy, indicating no inhibition of microorganism growth. These observations indicate that unbound amine in concentrations sufficient to inhibit growth of the microorganism was not present in the aqueous solution outside the dialysis bag. The dearth of unbound amine outside the dialysis bag indicates that a significant amount of amine was not migrating out of the amine-polymer into the fluid in the dialysis bag. The lack of unbound amine in the dialysis bag coupled with the inhibition of microorganism growth in the fluid in the dialysis bag indicates that the mechanism of inhibition of microorganism growth results in binding of the amine at the AG Polymer surface to portions of the microorganism. The fact that the amine-polymer composition inhibits growth of microorganisms after repeated antimicrobial activity tests indicates that the amine bound to portions of the microorganism is no longer retained at the amine-polymer composition's surface.

1-63. (canceled)

64. A composition comprising:
a quantity of polymer;
a quantity of amine;
wherein the quantity of polymer comprises a first number of monomer units;
wherein a number of (Si(CH3)2O) monomer units in the quantity of polymer is less than 99 percent of the first number of monomer units;
wherein the quantity of polymer comprises one or more polymers selected from the group consisting of silicones, polyurethanes, other elastomers, polyolefins, polyamides, polyesters, polyethers, and blends and copolymers of any one or more of the foregoing;
wherein the quantity of amine consists one or more amines, and combinations thereof;
wherein each one of the one or more amines is selected from the group of primary, secondary, and tertiary amines comprising three R groups;
wherein, for each one of the one or more amines selected from the group of primary, secondary, and tertiary amines,
(1) a first one of the three R groups is a hydrocarbon group of 6 to 20 carbons,
(2) a second one of the three R groups is a member selected from the group consisting of a hydrogen and a hydrocarbon group consisting of 1 to 8 carbons, and
(3) a third one of the three R groups is a member selected from the group consisting of a hydrogen and a hydrocarbon group consisting of 1 to 8 carbons;
wherein the composition occupies a composition volume delimited by a composition surface defining a composition surface area;
wherein the quantity of amine has a weight between 0.001 and 10 milligrams per square centimeter times the composition surface area;
wherein the quantity of amine is inhomogeneously distributed in the composition volume; and
wherein the quantity of amine is neither covalently nor ionically bonded to the quantity of polymer.

65. The composition of claim 64 wherein the quantity of amine has a weight between 0.032 to 1.280 milligrams per square centimeter times the composition surface area.

66. The composition of claim 64 wherein the quantity of amine comprises an amine having the first one of the three R groups being a hydrocarbon group of 10 to 17 carbons.

67. The composition of claim 64 wherein the quantity of polymer comprises one or more polymers selected from the group consisting of polyurethanes, polyolefins, polyamides, polyethers, elastomers other than polydimethylsiloxane (PDMS), and blends and copolymers of any one or more of the foregoing.

68. The composition of claim 64 wherein the quantity of amine comprises an amine that is a tertiary amine.

69. The composition of claim 64 wherein the quantity of amine comprises an amine having the second and third ones of the three R groups being methyl groups.

70. The composition of claim 64 wherein the quantity of amine comprises N,N-dimethyltetradecylamine.

71. The composition of claim 64 wherein the weight percent of the quantity of amine to the quantity of polymer is between 0.046 to 1.84.

72. The composition of claim 64 wherein the composition surface area defines a first composition surface area and a second composition surface area;
wherein the first composition surface area opposes the second composition surface area;
wherein a upper region defines an upper region volume extending between the first composition surface area and the second composition surface area to an upper region bottom;
wherein the upper region has an upper region amine density defined as weight of the quantity of amine in the upper region volume;
wherein a lower region defines an lower region volume extending between the first composition surface area and the second composition surface area from the upper region bottom to the lower region bottom;
wherein the lower region has an lower region amine density defined as weight of the quantity of amine in the lower region volume; and
wherein the upper region amine density is greater than the lower region amine density.

73. The composition of claim 64 wherein the composition comprises a region having tubular shape.

74. The composition of claim 64 wherein the composition comprises a region having a sheet shape.

75. The composition of claim 64 further comprising:
a filler of at least 5% by weight.

76. An implantable medical device, comprising the composition of claim 64.

77. A catheter, comprising the composition of claim 64.

78. A product that is any one of a tube, a catheter, a drainage bag, and a prosthetic, wherein the product comprises the composition of claim 64.

79. A composition comprising a quantity of polymer and a quantity of amine, formed by a water-rich process comprising:
preparing a mixture comprising (1) an aqueous solution comprising more than fifty percent by volume of water and (2) amine comprising the quantity of amine; and
exposing the quantity of polymer to the mixture;
wherein the quantity of polymer comprises one or more polymers selected from the group consisting of silicones, polyurethanes, other elastomers, polyolefins, polyamides, polyesters, polyethers, and blends and copolymers of any one or more of the foregoing;
wherein the quantity of amine consists of one or more amines, and combinations thereof;
wherein each one of the one or more amines is selected from the group of primary, secondary, and tertiary amines comprising three R groups;
wherein, for each one of the one or more amines selected from the group of primary, secondary, and tertiary amines,
(1) a first one of the three R groups is a hydrocarbon group of 6 to 20 carbons,
(2) a second one of the three R groups is a member selected from the group consisting of a hydrogen and a hydrocarbon group consisting of 1 to 8 carbons, and
(3) a third one of the three R groups is a member selected from the group consisting of a hydrogen and a hydrocarbon group consisting of 1 to 8 carbons;
wherein the composition occupies a composition volume delimited by a composition surface defining a composition surface area;
wherein the quantity of amine has a weight between 0.001 and 10 milligrams per square centimeter times the composition surface area;
wherein the quantity of amine is inhomogeneously distributed in the composition volume; and
wherein the quantity of amine is neither covalently nor ionically bonded to the quantity of polymer.

80. The composition of claim 79, further comprising forming the mixture into an emulsion of the quantity of amine in the aqueous solution prior to exposing the quantity of polymer to the mixture.

81. An implantable medical device, comprising the composition of claim 79.

82. A catheter, comprising the composition of claim 79.

83. A product that is any one of a tube, a catheter, a drainage bag, and a prosthetic implant, wherein the product comprises the composition of claim 79.
